(19) United States
(12) Patent Application Publication
(10) Pub. No.: US 2003/0039669 A1
(43) Pub. Date: Feb. 27, 2003

(54) Use of a composition containing an effective quantity of at least one chelating agent for partially or totally reducing the symptoms associated with histamine release in the organism

(75) Inventors: Olivier De Lacharriere, Paris (FR); Roland Jourdain, Meudon la Foret (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 10/127,566

(22) Filed: Apr. 23, 2002

(30) Foreign Application Priority Data
Apr. 23, 2001 (FR) 0105458

(51) Int. Cl. A61K 38/40; A61K 7/00

(52) U.S. Cl. 424/401; 514/6; 514/55

(57) ABSTRACT

Use to partially or totally reduce the symptoms associated with histamine release in the organism, particularly pruritus and erythema, which comprises the topical application of a cosmetic and/or dermatological and/or hygiene composition containing an effective quantity of at least one ion chelating agent, said composition being essentially free of flavones, flavonones and/or flavonoids, and free of sequestring agents such as polyaspartic acid and its salts, cellulose derivatives and copolymers containing maleic or hydrosuccinic acid as monomeric building blocks and salts thereof.

The present invention relates to the use of a composition containing an effective quantity of at least one chelating agent for partially or totally eliminating the symptoms associated with histamine release, particularly erythema and pruritus.

Histamine is an amine derived from histidine, present in animal tissue. Histamine is a chemical mediator of phenomena such as gastric secretions, allergies, etc.

A chemical mediator should be understood to mean a substance released by a cell and involved in a process in the organism (nerve conduction, inflammation). Histamine is released in the organism from mastocytes, which are connective tissue cells which secrete chemical substances participating in immune reactions and blood coagulation, and are involved in allergic phenomena.

The causes of histamine release from mastocytes are varied. There are immunological causes (reactive hypersensitivity, activation of complement C3a, IgG antireceptor to IgE with high Fc_{ε}RIa affinity) and non-immunological causes (drugs, scratches).

The consequences of the release of histamine are oedema, erythema and pruritus (Lewis's triad).

The applicant has discovered that topical application of a composition containing an ion chelating agent (EDTA) reduces the symptoms caused by an application of histamine by iontophoresis, and in particular pruritus and erythema. This test is a good model for evaluating the antipruritic and anti-erythema activity of a topical composition.

The Japanese patent JP-9323920 discloses a detergent composition containing an ester sulfate as surfactant, a cosurfactant, a cationic bactericide and a chelating agent used for cleaning the skin and hair, showing an antibacterial, anti-dandruff and antipruritic activity.

However, this document does not relate to a method for partially or totally eliminating the symptoms associated with histamine release. In addition, there is no mention of the antihistaminic activity of the chelating agent.

The object of the present invention is thus the use of a cosmetic and/or dermatological and/or hygiene composition containing an effective quantity of at least one ion chelating agent for partially or totally eliminating the symptoms associated with histamine release, in particular erythema and pruritus, said composition being essentially free of flavones, flavonones and/or flavonoids, and free of sequestring agents such as polyaspartic acid and its salts, cellulose derivatives and copolymers containing maleic or hydrosuccinic acid as monomeric building blocks and salts thereof.

By ion chelating agent should be understood chemical or biological compounds (proteins, peptides, etc.) with the ability to sequester ions (anions or cations).

Examples of chemical chelating agents include:

aminotrimethyl phosphonic acid,

β-alanine diacetic acid,

citric acid,

cyclodextrin,

cyclohexanediamine tetracetic acid,

diethylenetriamine pentamethylene phosphonic acid,

diethanolamine N-acetic acid,

ethylene diamine tetracetic acid (EDTA or YH₄) and its sodium (YH₃Na, Y₂H₂Na₂, YHNa₃ and YNa₄), potassium (YH₃K, Y₂H₃K₃ and YK₄), calcium disodium, and diammonium salts and its salts with triethanolamine (TEA-EDTA),

etidronic acid,

galactanic acid,

hydroxyethyl ethylenediamine tetracetic acid (HEDTA) and its trisodium salt,

L-tartaric acid,

gluconic acid,

glucuronic acid,

nitrilotriacetic acid (NTA) and its trisodium salt,

pentetic acid,

phytic acid,

ribonic acid,

diammonium citrate,

disodium azacycloheptane diphosphonate,

disodium pyrophoshate,

hydroxypropyl cyclodextrin,

methyl cyclodextrin,

pentapotassium triphosphate,

pentasodium aminotrimethylene phosphonate,

pentasodium ethylenediamine tetramethylene phosphonate,

pentasodium pentetate,

pentasodium triphosphate,

potassium citrate,

potassium EDTMP,

sodium EDTMP,

sodium chitosan methylene phosphonate,

sodium hexametaphosphate,

sodium metaphosphate,

potassium polyphosphate,

sodium polyphosphate,

sodium trimetaphosphate,

sodium dihydroxyethylglycinate,

potassium gluconate,

sodium gluconate,

sodium glucopeptate,

sodium glycereth-1 polyphosphate,

tetrapotassium pyrophosphate,

triethanolamine polyphosphate (TEA),

tetrasodium pyrophosphate,

trisodium phosphate,

potassium triphosphonomethylamine oxide,

sodium metasilicate,

sodium phytate,

sodium polydimethylglycinophenolsulfonate,

tetrahydroxyethyl ethylene diamine,

tetrahydroxypropyl ethylene diamine,

tetrapotassium etidronate,

tetrasodium etidronate,

tetrasodium iminodisuccinate,

trisodium ethylenediamine disuccinate,

ethanolamine N,N-diacetic acid,

disodium acetate,

dimercaprol,

deferoxamine,

Zylox, iron chelating agent disclosed and claimed in the international patent application WO 94/61338,

this list being non-limiting.

A preferred chemical chelating agent according to the invention is selected from ethylenediamine tetracetic acid (EDTA) and its sodium, potassium, calcium disodium, diammonium, and triethanolamine salts (TEA-EDTA), hydroxyethyl ethylenediamine tetracetic acid (HEDTA) and its trisodium salt, and their mixtures.

Examples of biological chelating agents include metallothionein, transferrin, calmodulin, chitosan and their derivatives, this list being non-limiting.

The chelating agent is present in the composition used in the method according to the invention in a quantity representing from 10⁻⁶ to 10% by weight, and preferably from 0.01 to 5% by weight of the total weight of the composition.

The composition used in the method according to the invention may additionally contain at least one liquid or solid fatty phase.

In the context of this application, liquid fatty phase should be understood to mean a liquid fatty phase at ambient temperature (25° C.) composed of one or more fatty bodies which are liquid at ambient temperature, also called oils, and are intercompatible.

The oils of the fatty phase of the compositions according to the invention may be polar or non-polar, volatile or non-volatile oils, selected from the oils conventionally used in cosmetics.

The polar oils include the hydrocarbon oils containing ester, ether, acid or alcohol functions or their mixtures, such as for example:

the hydrocarbon oils with a high content of triglycerides consisting of esters of fatty acids and glycerol, of which the fatty acids may have varying chain lengths and may be linear or branched, saturated or unsaturated; these oils particularly include wheat germ, corn, sunflower, and shea oils, sweet almond, macadamia, apricot, soya, rapeseed, cotton, alfalfa, poppy, pumpkin, sesame, marrow, avocado, hazelnut, grapeseed, blackcurrant, evening primrose, millet, barley, quinoa, olive, rye, safflower, bancoulier, passion flower, rose hip oils and castor oil; or triglycerides of caprylic/capric acids such as those marketed by the Company Stearineries Dubois or those marketed under the trade names Miglyol 810, 812 and 818 by the Company Dynamit Nobel,

the synthetic oils of formula R¹COOR² in which R¹ represents a linear or branched higher fatty acid residue, containing from 7 to 19 carbon atoms, and R² represents a branched hydrocarbon chain containing from 3 to 20 carbon atoms, such as for example Purcellin oil (ketostearyl octanoate), isononyl isononanoate, the alkyl benzoates,

the synthetic esters and ethers such as isopropyl myristate, 2-ethylhexyl palmitate, the octanoates, decanoates or ricinoleates of alcohols or polyalcohols,

the hydroxyl esters such as isostearyl lactate, diisostearyl malate, and the pentaerythritol esters;

the C₈ to C₂₆ fatty alcohols such as oleic alcohol ; and

their mixtures.

The apolar oils include:

the silicone oils which may be volatile or non-volatile, linear or cyclic, liquid at ambient temperature, such as the polydimethylsiloxanes (PDMS) containing alkyl, alkoxy or phenyl groups, within or at the end of the silicone chain and having from 2 to 24 carbon atoms; the phenylated silicones, such as the phenyl trimethiocones, the phenyl dimethicones, the phenyl trimethylsiloxy diphenylsiloxanes, the diphenyidimethicones, the diphenyl methyldiphenyl trisiloxanes, the 2-phenylethyl trimethylsiloxysilicates,

the hydrocarbons or fluorinated hydrocarbons, which may be linear or branched and of synthetic or inorganic origin, such as the paraffin oils (for example the isoparaffins), and the aliphatic hydrocarbons (for example isododecane), and their derivatives, vaseline, the polydecenes, hydrogenated polyisobutene such as parleam, squalane, and their mixtures.

The oils of the fatty phase are preferably apolar oils of the inorganic or synthetic hydrocarbon type, selected in particular from hydrocarbons, especially alkanes, such as parleam oil, the isoparaffins including isododecane, squalane and their mixtures.

In the context of this application, solid fatty phase should be understood to mean a lipophilic fatty compound, solid at ambient temperature (25° C.), with a melting point greater than 40° C. and up to 200° C., in other words a wax.

The waxes are those generally used in the cosmetic or dermatological fields. They are mainly of natural origin such as beeswax, Carnauba, Candellila, Ouricoury or Japanese waxes, cork or sugarcane fibres, paraffin waxes, lignite, microcrystalline waxes, lanolin wax, Montan wax, the ozokerites, the hydrogenated oils such as hydrogenated jojoba oil, but also of synthetic origin such as the polyethylene waxes produced by the polymerization of ethylene, the waxes obtained by the Fischer-Tropsch synthesis, the esters of fatty acids and the glycerides solid at 40° C., the silicone waxes such as the alkyl, alkoxy and/or esters of poly(di)methylsiloxane solid at 40° C.

As is known, the cosmetic composition used in the method according to the invention may also contain adjuvants conventionally used in cosmetics such as water, optionally thickened or gelified by a thickening agent or a gelling agent of the aqueous phase, antioxidants, essential oils, preservatives, neutralizing agents, liposoluble polymers, fillers, perfumes, emulsifying agents, gelling agents, filters, odour absorbers and colorants.

The quantities of these different adjuvants are those conventionally used in cosmetics, for example from 0.01% to 10% of the total weight of the composition. These adjuvants, depending on their properties, may be introduced into the fatty phase, the aqueous phase and/or the lipid globules.

Emulsifying agents which may be used in the invention include for example glycerol stearate, polysorbate 60 and the mixture of PEG-6/PEG-32/Glycol Stearate marketed under the trade name Tefose® 63 by the Company Gattefosse.

Solvents which may be used in the invention include the lower alcohols, particularly ethanol, isopropanol and propylene glycol.

Hydrophilic gelling agents which may be used in the invention include the carboxyvinylic polymers (carbomer), the acrylic copolymers such as the copolymers of acrylates/alkyl acrylates, the polyacrylamides, the polysaccharides such as hydroxypropylcellulose, the natural gums and the clays.

Lipophilic gelling agents which may be used in the invention include the modified Flays such as the bentones, the metal salts of fatty acids such as the aluminium stearates and hydrophobic silica, ethylcellulose, polyethylene.

The composition used in the method according to the invention may contain other hydrophilic active ingredients such as the proteins or protein hydrolysates, the amino acids, the polyols, urea, allantoin, the sugars and sugar derivatives, the hydrosoluble vitamins, plant extracts and the hydroxy-acids.

Lipophilic active ingredients may include retinol (vitamin A) and its derivatives, tocopherol (vitamin E) and its derivatives, the essential fatty acids, the ceramides, the essential oils, salicylic acid and its derivatives.

The composition used in the method according to the invention may also combine at least one metal ion chelating agent with other active ingredients specifically intended for the control and/or treatment of skin diseases. These active ingredients may include for example:

the agents modifying the differentiation and/or the proliferation and/or the pigmentation of the skin such as retinoic acid and its isomers, retinol and its esters, vitamin D and its derivatives, the oestrogens such as oestradiol, kojic acid or hydroquinone,

the antibacterials such as clindamycin phosphate, erythromycin or the antibiotics of the tetracycline class;

the antiparasitics, in particular metronidazole, crotamiton or the pyrethrinoids;

the antifungal agents, in particular the compounds belonging to the imidazole class such econazole, ketoconazole or miconazole or their salts,

the polyene compounds, such as amphotericin B, the compounds of the allylamine family such as terbinafine, or octopiroxy;

the antiviral agents such as acyclovir;

the steroidal anti-inflammatories, such as hydrocortisone, betamethasone valerate or clobetasol propionate, or the non-steroidal anti-inflammatories such as ibuprofene and its salts, diclofenac and its salts, acetylsalicyclic acid, acetaminophene or glycyrrhetinic acid;

the anaesthetics such as lidocaine chlorhydrate and its derivatives;

the antipruritic agents such as thenaldine, trimeprazine or cyproheptadine;

the keratolytic agents such as the alpha- and beta-hydroxycarboxylic or beta-ketocarboxylic acids, their salts, amides or esters and more particularly the hydroxy-acids such as glycolic acid, lactic acid, salicylic acid, citric acid and in general the fruit acids, and 5-n-octanoylsalicyclic acid;

the anti-free radical agents, such as alpha-tocopherol or its esters, the dismutase superoxides, certain metal chelating agents or ascorbic acid and its esters;

the anti-seborreics such as progesterone;

the anti-dandruff agents such as octopirox or zinc pyrithione;

the anti-acne agents such as retinoic acid or benzoyl peroxide.

Colorants which may be used in the invention include the lipophilic colorants, the hydrophilic colorants, the pigments and the nacres normally used in cosmetic or dermatological compositions, and their mixtures. This colorant is generally used at a concentration of from 0.01 to 40% of the total weight of the composition, preferably from 5 to 25%.

The liposoluble colorants are, for example, Sudan red, DC Red 17, DC Green 6, carotene, soya oil, Sudan brown, DC Yellow 11, DC Violet 2, DC orange 5, quinoline yellow. They may represent from 0 to 20% of the weight of the composition and preferably from 0.1 to 6%.

The pigments may be white or coloured, inorganic and/or organic, coated or not.

The inorganic pigments include titanium dioxide, optionally surface-treated, zirconium or cerium oxides, and the iron and chromium oxides, manganese violet, ultramarine blue, chromium hydrate and ferric blue.

The preferred inorganic pigments are the iron oxides, especially red iron oxide, yellow iron oxide, red and yellow iron oxide, brown iron oxide, black iron oxide, and titanium dioxide.

The organic pigments include:

carbon black,

the pigments of type D&C, such as D&C Red No 36, and

the lakes based on cochineal carmine, barium, strontium, calcium such as D&C Red No 7 calcium lake, aluminium, such as D&C Red No 27 aluminium lake, D&C Red No 21 aluminium lake, FD&C Yellow No 5 aluminum lake, FD&C Yellow No 6 aluminium lake, D&C Red No 7 and FD&D Blue No 1.

The pigments may represent from 0 to 40% of the total weight of the composition, preferably from 2 to 25%.

The nacre pigments may be selected from the white nacre pigments, such as mica coated with titanium or bismuth oxychloride, the coloured nacre pigments such as titanium mica with iron oxides, titanium mica with in particular ferric blue or chromium oxide, titanium mica with an organic pigment of the type mentioned above, and the nacre pigments based on bismuth oxychloride. They may represent from 0 to 20% of the total weight of the composition and preferably from 0.1 to 15%.

Depending on the method of administration, the composition used in the method according to the invention may be in any pharmaceutical form normally used for topical application, intended especially for the cosmetic and/or dermatological and/or hygiene fields.

The composition used in the method according to the invention may be applied onto the skin (on any part of the body), onto the scalp or onto the mucosa (buccal, jugal, gingival and conjunctival).

For topical application to the skin, the composition may be in particular in the form of an aqueous or oily solution or a dispersion of the lotion or serum type, or emulsions of liquid or semi-liquid consistency of the milk type, obtained by dispersion of a fatty phase in an aqueous phase (H/E) or the inverse (E/H), or suspensions or emulsions of soft consistency of the cream type or an aqueous or anhydrous gel, or microcapsules or microparticles, or vesicular dispersions of the ionic and/or non-ionic type. These compositions are prepared by the usual methods.

The composition used in the method according to the invention may also be used on the scalp in the form of aqueous, alcoholic or hydro-alcoholic solutions, or in the form of creams, gels, emulsions, foams or in the form of compositions for aerosols additionally containing a propellant agent under pressure.

The quantities of the different constituents in the compositions used in the method according to the invention are those conventionally used in the fields concerned.

These compositions particularly comprise creams for cleansing, protection, treatment or care for the face, hands, feet, the major skin folds, or for the body (for example day creams, night creams, make-up removal creams, foundation creams, sunscreen creams), liquid foundations, cleansing milks, body milks for protection or care, sunscreen milks, lotions, gels or foams for skin care, such as cleansing lotions, sunscreen lotions, artificial tanning lotions, bath compositions, deodorant compositions, containing a bactericide, after-shave gels or lotions, depilatory creams, compositions against insect bites, painkilling compositions, compositions for treating skin disorders such as eczema, rosacea, psoriasis, the lichens, and severe pruritus.

The compositions used in the method according to the invention may also comprise solid preparations comprising cakes of soap or other cleansing products.

The compositions may also be packaged in the form of compositions for aerosols additionally containing a propellant agent under pressure.

When the composition used in the method according to the invention is an emulsion, the proportion of the fatty phase may be from 5% to 80% by weight, and preferably from 5% to 50% by weight with respect to the total weight of the composition.

The emulsifying agent and co-emulsifying agent are present in the composition in a proportion of from 0.3% to 30% by weight, and preferably from 0.5 to 20% by weight with respect to the total weight of the composition. The emulsion may, in addition, contain lipid vesicles.

When the composition used in the method according to the invention is a solution or an oily gel, the fatty phase may represent more than 90% of the total weight of the composition.

The methods according to the invention may be used by applying the compositions such as those defined above, according to the normal technique for using these compositions. For example: application of creams, gels, serums, lotions, cleansing milks or sunscreen compositions onto the skin or onto dry hair, application of a hair lotion onto wet hair, shampoos, or application of dentifrice onto the gums.

The following examples and compositions illustrate the invention without in any way limiting it. In the compositions, except where otherwise stated, the proportions are percentages by weight.

### EXAMPLE

Functional In vivo Test for the Effect of EDTA Against Erythema and Pruritus Induced by Application of Histamine by Iontophoresis

A functional in vivo test in humans was performed to demonstrate, on a population with normal skin, the properties of EDTA against the symptoms induced by application of histamine by iontophoresis on the forearm.

Subjects

12 healthy volunteers, of female sex, aged from 18 to 45, of phototype I to IV,

Products Tested

chelating agent: salt of disodium ethylenediamine tetraacetate (EDTA),

cosmetic compositions according to the invention CM₁, CM₂ and CM₃ containing respectively 0.05%, 0.5% and 2% of EDTA and in the form of a gel.

cosmetic composition CM₀ not containing EDTA (vehicle of CM₁, CM₂ and CM₃) and in the form of a gel.

The compositions CM₀ to CM₃ are given in table 1.

TABLE 1

| | CM₀ | CM₁ | CM₂ | CM₃ |
|---|---|---|---|---|
| Methyl paraben | 0.2% | 0.2% | 0.2% | 0.2% |
| Disodium EDTA | 0% | 0.05% | 0.5% | 2% |
| Triethanolamine | 0.7% | 0.7% | 0.7% | 0.7% |
| Carbomer | 0.7% | 0.7% | 0.7% | 0.7% |
| Water | 98.4 | 98.35 | 97.9 | 96.4% |

The compositions CM₁, CM₂ and CM₃ were tested in single applications against the vehicle (composition CM₀) during 3 visits taking place at minimum intervals of 15 days.

visit 1: test of composition CM₁,

visit 2: test of composition CM₂,

visit 3: test of composition CM₃.

Methodology

This was a prospective, monocentric, double-blind, randomized study, with the vehicle (composition CM₀) as control, with an intra-individual comparison (right forearm/left forearm).

The cosmetic composition CM₀ was applied at t=0 onto one of the forearms and one of the cosmetic compositions CM₁, CM₂ or CM₃ onto the other forearm.

Histamine was applied at t=30 minutes by iontophoresis onto the treated areas.

Subsequently, between t=35 minutes and t=50 minutes, the following were evaluated:

the area (in cm²) of erythema induced by the histamine at t=35, 40, 45, 50 minutes

the pruritus induced by the histamine at the same times, i.e. at t=35, 40, 45, 50 minutes, using the following scale:

0=no sensation

1=light or doubtful

2=moderate

3=significant

Results

1) Area of Erythema

The results are given in terms of the average area (in cm²) of induced erythema over the whole period, and are shown in table 2.

TABLE 2

| | | Average area of | |
|---|---|---|---|
| | | erythema (cm²) ± | |
| | | standard deviation of | Comparison |
| | | the mean | effects CM₁/CM₀ |
| Visit 1 | CM₀ vehicle | 7.77 ± 0.69 | reduction |
| | (0% EDTA) | | of 4% |
| | CM₁ | 7.45 ± 0.77 | |
| | (0.05% EDTA) | | |
| Visit 2 | CM₀ vehicle | 10.57 ± 1.11 | reduction |
| | (0% EDTA) | | of 11% |
| | CM₂ | 9.41 ± 0.92 | |
| | (0.5% EDTA) | | |
| Visit 3 | CM₀ vehicle | 7.75 ± 1.07 | reduction |
| | (0% EDTA) | | of 26% |
| | CM₃ | 5.75 ± 0.83 | |
| | (2% EDTA) | | |

A reduction of the extent of the induced erythema was observed when the compositions CM₁, CM₂ and CM₃, containing respectively 0.05%, 0.5% and 2% of EDTA, were applied.

The reduction of the extent of the erythema was proportional to the concentration of EDTA.

2) Average Pruritus Score

The results are given in terms of the average pruritus score over the whole period, and are shown in table 3.

A reduction of about 50% of the pruritus was observed for the compositions CM₁ and CM₂.

TABLE 3

| | | Average | |
|---|---|---|---|
| | | pruritus score ± | |
| | | standard deviation of | Comparison |
| | | the mean | effects CM₁/CM₀ |
| Visit 1 | CM₀ | 0.79 ± 0.17 | reduction |
| | Vehicle | | of 58% |
| | (0% EDTA) | | |
| | CM₁ | 0.35 ± 0.07 | |
| | (0.05% EDTA) | | |
| Visit 2 | CM₀ | 0.44 ± 0.15 | reduction |
| | vehicle | | of 47% |
| | (0% EDTA) | | |
| | CM₁ | 0.23 ± 0.08 | |
| | (0.5% EDTA) | | |
| Visit 3 | CM₀ | 0.19 ± 0.08 | Intensity of |
| | vehicle | | pruritus too low |
| | (0% EDTA) | | in the control |
| | CM₁ | 0.19 ± 0.08 | group |
| | (2% EDTA) | | test |
| | | | discarded |

1. Use to partially or totally reduce the symptoms associated with histamine release in the organism, particularly pruritus and erythema, which comprises the topical application of a cosmetic and/or dermatological and/or hygiene composition containing an effective quantity of at least one ion chelating agent, said composition being essentially free of flavones, flavonones and/or flavonoids, and free of sequestring agents such as polyaspartic acid and its salts, cellulose derivatives and copolymers containing maleic or hydrosuccinic acid as monomeric building blocks and salts thereof.

2. Use according to claim 1, characterized in that the chelating agent is a chemical chelating agent selected from ethylenediamine tetracetic acid (EDTA) and its sodium, potassium, calcium disodium, diammonium, and triethanolamine (TEA-EDTA) salts, hydroxyethyl ethylenediamine tetracetic acid (HEDTA) and its trisodium salt and their mixtures.

3. Use according to claim 1 or 2, characterized in that the chelating agent is a biological chelating agent selected from metallothionein, transferrin, calmodulin, chitosan and its derivatives and their mixtures.

4. Use according to any of the preceding claims, characterized in that the chelating agent is present in the composition in a quantity representing from 10⁻⁶ to 10% by weight, preferably from 0.01 to 5% by weight of the total weight of the composition.

5. Use according to any of the preceding claims, characterized in that the composition contains at least one liquid or solid fatty phase.

6. Use according to claim 5, characterized in that the fatty phase is liquid and contains at least one oil selected from the hydrocarbon oils, the plant oils, the animal oils, the synthetic oils, the silicone oils and the fluorinated oils.

7. Use according to claim 5, characterized in that the fatty phase is solid and contains at least one wax selected from the natural waxes such as beeswax, Carnauba wax, paraffin wax, the esters of fatty acids, the fatty alcohols and the silicone waxes.

8. Use according to any of the preceding claims, characterized in that the composition contains at least one cosmetic active ingredient.

9. Use according to any of the preceding claims, characterized in that the composition contains at least one additive selected from water, the antioxidants, the essential oils, the preservatives, the neutralizing agents, the liposoluble polymers, the fillers, the perfumes, the emulsifying agents, the gelling agents, the filters, the odour absorbers and the colorants.

10. Use according to any of the preceding claims, characterized in that the composition is in the form of an aqueous or oily solution or dispersion of the lotion or serum type, for topical application to the skin.

11. Use according to any of claims 1 to 9, characterized in that the composition is in the form of an emulsion of liquid or semi-liquid consistency of the milk type, obtained by dispersion of a fatty phase in an aqueous phase (H/E) or of an aqueous phase in a fatty phase (E/H), for topical application to the skin.

12. Use according to any of claims 1 to 9, characterized in that the composition is in the form of an suspension or emulsion of soft consistency of the cream or aqueous or anhydrous gel type, for topical application to the skin.

13. Use according to any of claims 1 to 9, characterized in that the composition is in the form of aqueous, alcoholic or hydroalcoholic solutions, for application to the scalp.

14. Use according to any of claims 1 to 9, characterized in that the composition is in the form of creams, gels, emulsions or mousses, for application to the scalp.

15. Use according to any of claims 1 to 9, characterized in that the composition is in the form of an aerosol and contains a propellant agent under pressure, for application to the scalp.
